(19) United States
(12) Patent Application Publication
(10) Pub. No.: US 2022/0134210 A1
(43) Pub. Date: May 5, 2022

(54) Device with a Display Assembly for Displaying Content with a Holographic Effect

(71) Applicant: etone Motion Analysis GmbH, Berlin (DE)

(72) Inventors: Calin-Laurentiu Popescu, Berlin (DE); Valerie Bures-Bönström, Berlin (DE)

(21) Appl. No.: 17/106,376

(22) Filed: Nov. 30, 2020

Related U.S. Application Data
(63) Continuation of application No. 17/086,708, filed on Nov. 2, 2020.

(51) Int. Cl. A63B 71/06; G02B 7/182; G16H 40/63; G02B 27/14; G16H 20/30

(52) U.S. Cl. CPC A63B 71/0622; G02B 7/182; G16H 40/63; G06F 3/147; G16H 20/30; A63B 2071/0658; G02B 27/144

(57) ABSTRACT

A device including a mirror which reflects incident light from a user facing a front surface of the mirror to present a reflected image of the user and which transmits incident light from a rear surface of the mirror; and at least one display arranged behind the mirror for displaying content superimposed on at least a portion of the reflected image of the user. The display is configured to display at least one visual element of the content on a black toned background. The display and the mirror are configured to create a holographic effect for the at least one visual element by an additional projection of the at least one visual element on the mirror, and, for generating the additional projection, a front side of the display at which the content is displayed is arranged at a predetermined minimum distance to the rear surface of the mirror and the mirror has a predetermined transparency-opacity ratio.

### CROSS-REFERENCE TO RELATED APPLICATION

This application is a Continuation-In-Part of U.S. patent application Ser. No. 17/086,708 filed Nov. 2, 2020, the disclosure of which is hereby incorporated by reference in its entirety.

### BACKGROUND OF THE INVENTION

### Field of the Invention

The present solution relates to a device comprising at least one display for displaying content to user facing a front side of the device.

### Description of Related Art

Fitness devices which are to be used in gyms or at home and which include at least one display for displaying exercise content to a user become more and more popular. The exercise content may be a video retrieved from a local memory of the fitness device or from a memory of a computing device connected to the fitness device via a wired or wireless network. Alternatively or additionally exercise content may be provided as a stream, for example via the Internet. Based on the exercise content the user is instructed how to perform different fitness exercises. In order to provide a new and motivating experience to the user and providing the user with sufficient details on how to carry out specific exercise properly, the at least one display of the fitness device may be rather large and held by a frame structure of the fitness device having more than 1 metre in length and more than 40 cm in width.

In this context fitness devices are also known which include a semi-reflective mirror arranged in front of the at least one display. Such a mirror may reflect incident light from a user facing a front surface of the mirror to present a reflected image of the user. Further, the mirror may transmit incident light from a rear surface of the mirror so that exercise content displayed via the at least one the display is superimposed on at least a portion of the reflected image of the user. Thereby, the user may see his/her reflected image in combination with a visual element, like an image of a trainer or avatar in the exercise content, so that the user may easily notice whether a specific exercise is performed properly or not.

In addition or as an alternative to a mirror the fitness device may be equipped with camera configured to capture the user while exercise content is displayed via the at least one display in order to provide electronic feedback on how well the user is the following the instructions of the exercise content and performs an exercise displayed to the user. The user may also receive visual and/or acoustic feedback on her/his current training.

It is an object of the present solution to provide for a device, in particular a fitness device which may provide for an improved user experience, for example, by an enhanced usability and/or an improved user interface for controlling operations at the device.

### SUMMARY OF THE INVENTION

According to a first aspect, a device is proposed which comprises a mirror which reflects incident light from a user facing a front surface of the mirror to present a reflected image of the user and which transmits incident light from a rear surface of the mirror. Additionally, the device comprises at least one display arranged behind the mirror for displaying content superimposed on at least a portion of the reflected image of the user. The at least one display of the device is configured to display at least one visual element of the content on a black toned background, wherein the display and the mirror are further configured to create a holographic effect for the at least one visual element by an additional projection of the at least one visual element on the mirror. For generating the additional projection (a) a front side of the display at which the content is displayed is arranged at a predetermined minimum distance to the rear surface of the mirror, and (b) the mirror has a predetermined transparency-opacity ratio.

By arranging the display with its front side at a predetermined minimum distance to the rear surface of the mirror and providing the mirror with a predetermined transparency-opacity ratio a holographic effect may be achieved on the mirror (and thus at a front side of a display surface of the device) so that the display additionally projects the at least one visual elements on the mirror, in particular on the rear surface of the mirror. The minimum distance of the display to the rear surface of the mirror is based on a given transparency-opacity ratio of the mirror, thereby making sure that a duplicate of the visual element is projected on the mirror (directly in front of the representation of the visual element on the display), resulting in a holographic effect for the at least one visual element. The user may thus perceive the visual element with glowing and/or hollow effect improving visibility and visual impression of the displayed visual element. Using a black toned (and this for example a black or gray) background on which the visual element is a displayed combined with an arrangement of the display at a predetermined minimum distance to the rear surface of the mirror and a predetermined transparency-opacity ratio of the mirror may also generate a certain light reflection and refraction phenomena resulting in an favorable holographic effect for displayed visual elements. The proposed device may in particular be configured to display exercise content and therefore be used as a fitness device. The proposed solution shall however not be construed to be limited in this regard. In particular, the proposed solution may also be implemented in a device not related to fitness exercise, e.g., a device for videoconferencing.

In an exemplary embodiment, the at least one display is configured to display the at least one visual element (including just a portion of it) with a hue in the range of 0° to 345° in the HSL or HSV color space (HSL standing for “hue, saturation, lightness” and HSV standing for “hue, saturation, value” as alternative representations of the RGB color model).

Alternatively or additionally, the at least one display may be configured to display the black tone background with a black or gray color having a color code, according to or complying with the RGB color code, in which r=g=b. For a black background, r, g and b may of the same value and equal to 0. Exemplary gray backgrounds may be defined by rgb(220,220,220), rgb(211,211,211, rgb(192,192,192), rgb(169,169,169), rgb(128,128,128) or rgb(105,105,105).

Generally, the at least one display may be coupled to a computing device configured to calculate values for r, b and g (parameters) depending on the transparency-capacity ratio of the mirror. For example, for a given transparency-opacity ratio of the mirror the computing device—which may be a part of the device or just coupled to it—may define suitable values for displaying a corresponding black toned background so as to assure that the visual element displayed results in the desired projection on the mirror. The computing device may comprise at least one processor and a memory including instructions which, when executed by the at least one processor, cause the at least one processor to set the RGB values—or corresponding values in an alternative representation of the RGB color model—depending from a given transparency-opacity ratio of the mirror, namely based on a computationally implemented relationship between the color values and the transparency-opacity ratio.

In an exemplary embodiment, the predetermined minimum distance lies in the range of 1.5 millimeters to 7 millimetres. It has been found that a corresponding distance between the display and the mirror in certain configurations provides for a beneficial holographic effect agreeable to users of the device.

The transparency-opacity ratio of the mirror may lie in the range of ¼ to ¾. This means that a transparency of the (semi-reflective) mirror may range from 20% to 75% with the opacity respectively lying between 80% and 25%.

In an exemplary embodiment, the mirror may include a reflective film power coating for reflecting the incident light from the user facing the front side of the mirror. By using a reflective film powder coating the mirror may include a protective glass cover to which the reflective film powder is applied. Such a configuration might be beneficial with respect to costs and also with respect to facilitating manufacture of the mirror with a desired transparency-opacity ratio.

In an exemplary embodiment, the mirror has a thickness of more than 0.5 mm. The mirror is a thus at least 0.3 millimetres thick, wherein the thickness defines a distance between the front surface and the rear surface of the mirror. A certain minimum thickness of the mirror may be beneficial for achieving a desired holographic effect for visual elements displayed on the device.

Generally, the visual element may be just a “passive” elements, for example, relating to parts of a video or a stream instructing the user how a fitness exercise is to be performed or indicating to the user parameters to be taken into account when performing the fitness exercise, such as a number of repetitions or a threshold pulse which should not be exceeded. Additionally or alternatively, a displayed visual element may also relate to biometric data of the user. Further, also additionally or alternatively, a visual element may be “active” and may therefore define an interface element of a user interface of the device. In such an embodiment, the at least one visual element may thus define a region at which a user touching a touch-sensitive portion of the front surface of the mirror generates an actuation signal further processed by a computing device coupled to the at least one display. Touching a region of the mirror where visual element is displayed may thus trigger an operation event, e.g., in particular including representing a new or altered visual element.

For providing the device with a touch-sensitive portion a capacitive field may be applied to at least a portion of the mirror. The at least one touch-sensitive portion may generate of a user-interface of the device at a front side of the device.

In an exemplary embodiment, at least one camera may be included in the device for determining a position of the user. The device may then further comprise or is coupled to a computing device which is coupled to the at least one camera and the at least one display. The computing device comprises at least one processor and a memory including instructions which, when executed by the at least one processor, cause the at least one processor to (a) determine, based on image data from the at least one camera, the position of the user in front of the device, and (b) define at least one of an appearance, size and position of the at least one visual element for display at the at least one display depending on the determined position of the user. At least one of an appearance, a size and a position of the at least one visual element may thus depend on a position of the user, which is determined using at least one camera of the device. The computing device may generate visual element data, based on the determined position of the user, to be transmitted to the at least one display for having the visual element displayed with an appearance (including the presence of a certain holographic effect), size and/or position defined by the computing device. This approach may, for example, result in increasing a holographic effect, augmenting the visual element and/or adapting hue, color and/or brightness of a visual element depending on a determined position of the user. For example, the visual element may be displayed with an increased size and/or increased brightness in case the user is detected farer away from the device.

In an exemplary embodiment, the instructions, when executed by the at least one processor, may cause the at least one processor of the computing device to adjust at least one of the appearance, the size and the position of a visual element based on a change in the determined position of the user. A corresponding adjustment of at least one of the appearance, the size and the precision of the visual element may depend on a change in position exceeding at least one threshold value. Thereby, an adjustment of the corresponding parameters of the displayed visual element may be avoided due to every movement of the user and, for example, due to minor movements of the user. Just a change within a predefined tolerance range may be accepted without adjusting appearance, size and/or position of the visual element.

In an exemplary embodiment, the device comprises or is coupled to a computing device (in the latter case, for example, coupled to a computing device remote from the device and/or including at least one loud server), wherein the computing device comprises at least one processor and a memory including instructions which, when executed by the at least one processor, cause the at least one processor to pre-process content to be displayed by the at least one display of the device. A pre-processing of the content may comprise identifying at least one main visual element in the content for display and blackening out a background of the at least one identified main visual element in the content for display. In a corresponding embodiment, content to be displayed may be computationally analyzed by the at least one computing device for adapting the content in such a way that the desired holographic effect for the visual element is achieved. The identified at least one main visual element in the content for display may then constitute the at least one visual element, which is additionally projected on the mirror as a kind of duplicate for the desired holographic effect. For example, an algorithm implemented by the instructions may identify at least one key area, person or object within a content for display, such as a video or stream, which is mapped and identified as a main visual element (this may also include that several, separate main visual elements are identified). Based on the identified main visual element(s) the rest of an image in a frame of the video or stream may then blacked out in order to provide the main visual element(s) with a holographic effect based on the proposed configuration of the device. For example, a video or stream showing a person, in particular a trainer performing a fitness exercises, may be pre-processed in a way that the person is visible and the rest of the content surrounding the person is covered by a black toned background. In such a case, the person may be visible in a holographic form, which might be of great benefit for augmenting and presenting the person, not only as part of a video or a stream for exercise content but also for videoconferencing or in other situations where a certain visual element is considered to be of (more) importance compared to the rest of an information in the video or stream.

The device may comprise a frame structure which defines a continuous frame inside which the mirror is held. The mirror may thus be circumferentially completely encompassed by the frame. In an exemplary embodiment, the mirror may have a length of at least one 1.0 m and a width of at least 40 cm. Thereby, a user can see himself/herself in full height on the front surface of the mirror.

According to a second aspect of the proposed solution, a device comprises a mirror which reflects incident light from a user facing a front surface of the mirror to present a reflected image of the user and which transmits incident light from a rear surface of the mirror. The device additionally comprises at least one display for displaying the user interface superimposed on at least a portion of the reflected image of the user, wherein the at least one display is arranged behind the mirror at a predetermined minimum distance to the rear surface of the mirror. Further, the at least one display is configured to display a black toned background and to display the at least one interface element for the user interface the black toned background with at least a portion of the interface element having a hue in the range of 0° to 345° in the HSL of HSV color space. The display and the mirror may be further configured (and thus combined) to create a holographic effect for the at least one interface element by an additional projection of the at least one interface element on the mirror, for example on the rear surface of the mirror. As already exemplarily explained with respect to the first aspect, it has been found that the combination of at least one display arranged behind a mirror at a predetermined minimum distance and displaying information on a black toned background with a hue in the range of 0° to 345°, and thus not in black or gray, and for example with a certain brightness, may result in an advantageous holographic effect by presenting a duplicate of the visual element on the mirror. For an interface element of the user interface at the device are corresponding holographic effect may be of additional advantage regarding usability of a touch-sensitive front surface.

According to a third aspect of the proposed solution, a device comprises a mirror which reflects incident light from a user facing the front surface of the mirror to present a reflected image of the user and which transmits incident light from the rear surface of the mirror. In addition, the device comprises at least one display arranged behind the mirror for displaying at least one visual element superimposed on at least a portion of the reflected image of the user and being configured to display the at least one visual element on a black toned background. The at least one display and the mirror may be further configured to create a holographic effect for the at least one visual element by an additional projection of the at least one visual element on the mirror. For generating the additional projection, (a) the mirror has a predetermined transparency-capacity ratio, and (b) the at least one display is configured to display the black toned background with a black or gray color having a color code, according to the RGB color code, in which r=b=g. In particular, the RGB values r, b, and g may depend on the transparency-capacity ratio of the mirror.

The device may comprise an interface region including at least one of a connector for power supply, a connector for a wired network connection, a connector for a video or sound interface, like an HDMI plug or the like, and a switch, in particular a power switch. The interface region thus for example allows for connecting the device to a power supply, at least one computing device and/or a network via at least one cable. The interface region may for example be disposed at the back side of the frame structure so that the interface region is easily accessible at the back side.

The frame structure may define a continuous a frame inside which the mirror is held. In one embodiment, the mirror abuts on a bottom part and a top part of the frame as well as on two opposing side members connecting the bottom part and the top part. A mirror whose front surface extends over substantially the whole front side (except for the part of the frame structure at the circumference of the front side) is thus securely held within a frame of the frame structure which protects the mirror edges from damages and also facilitates assembly of the device. In an embodiment the frame of the frame structure has a thickness of less than 3 mm or even less than 2 mm. At the circumference of the mirror the frame is therefore rather slim and almost not perceivable at the front side.

In an exemplary embodiment, the front side of the device has a height and a width and the at least one display has a display height which is greater than one half of the height of the front side and has a display width which is greater than one half of the width of the front side. Accordingly, the at least one display of the proposed stand-alone device may be relatively large and may provide, for example, for a screen having a size of more than 34 inches, in particular more than 38 inches or 40 inches.

It its furthermore understood that also devices according to the second and third aspects may include several or all features of embodiments disclosed before and in the following with respect to the 1st aspect.

### BRIEF DESCRIPTION OF THE DRAWINGS

FIG. 1A is a perspective view on an embodiment of a proposed device with a view to a front side.

FIG. 1B is a perspective view of the device with a view to a back side.

FIG. 2A is a front view of the device.

FIG. 2B is back vow of the device.

FIG. 3 is a side view of the device illustrating dimensions of and angles between components of the device.

FIG. 4 is exploded view of the device.

FIG. 5 is an enlarged detail of the back side of the device showing a mounting region for a stand of the device.

FIG. 6 is an enlarged detail of a bottom part of a frame structure of the of device.

FIG. 7 is an enlarged detail of an upper portion of the device showing details of a (first) camera of the device and a power switch accessible at the front surface.

FIG. 8 is a perspective view on the frame structure of the device to which a display is mounted without showing a mirror and a back plate of the device.

FIG. 9 shows the display with its front surface.

FIG. 9B shows the display with its rear surface.

FIGS. 10A, 10B and 10C show different cross-sectional views of the device at an edge section.

FIG. 11A shows the device while displaying exercise content to a user in front of the device.

FIG. 11B shows an enlarged detail of the device illustrating a reflected image of the user and superimposed visual elements of the exercise content each displayed with a holographic effect.

FIG. 12 shows the device with exemplary fitness content displayed to another user in front of the device.

FIG. 13 shows details of a user interface of the device including interface elements displayed with a holographic effect.

### DESCRIPTION OF THE INVENTION

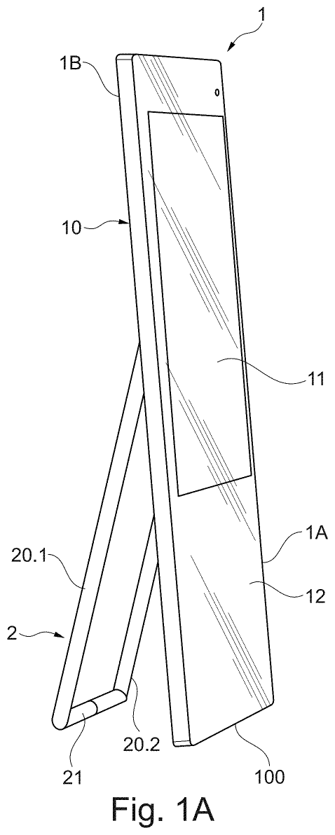

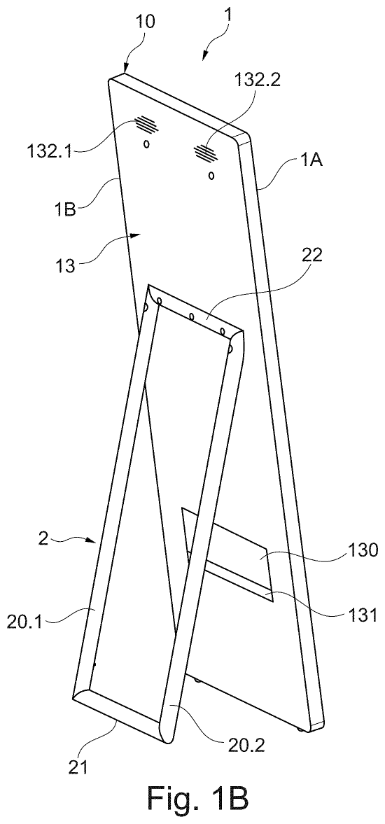

FIG. 1 illustrates an embodiment of a proposed device in the form of a stand-alone device 1 (sometimes also called a “smart mirror” or an “interactive screen”). The device 1 of the embodiment of FIG. 1 may be configured as a stand-alone fitness device to be used in a gym or at home (referred to as “fitness device” in the following). The fitness device 1 comprises a frame assembly including a frame structure 10, a display 11 and a mirror 12 (the display 11 and the mirror 12 defining a display assembly of the fitness device 1). The frame structure 10 defines a continuous frame surrounding the mirror 12. The mirror 12 is thus held within the frame of the frame structure 10. The display 11 is held behind the mirror 12. The display 11 includes a screen for displaying exercise content to a user standing in front of the fitness device 1. The frame assembly of the fitness device 1 defines a front side 1A and a back side 1B. The exercise content displayed via the display 11 may hence be watched by a user facing the front side 1A.

The mirror 12 may reflect incident light from the user facing a front surface of the mirror 12 to present a reflected image of the user. The mirror 12 may further transmit incident light from the rear surface of the mirror 12 so that exercise content displayed via the display 11 may be superimposed on at least a portion of the reflected image of the user. By superimposing exercise content on at least a portion of the selected image of the user the fitness device 1 may provide instant feedback on a performance of the user when imitating fitness exercises based on the displayed exercise content.

Alternatively or additionally, at least one camera may be part of the fitness device to capture images of the user during a fitness exercise in order to electronically provide feedback on the user's performance whether the user imitates a certain exercise properly and, e.g., to which extent.

The fitness device 1 as shown in FIG. 1A includes the display 11 having a screen with a diameter of at least 34 inches, for example more than 40 inches. Accordingly, the frame structure 10 defines a surface area at the front side of more than 0.5 m². For example, a substantially rectangular surface at the front side 1 measures more than 1.5 m in height and more than 0.4 m in width.

Given the size and weight of the fitness device 1 a secure and stable position of the fitness device 1 with its frame structure 10 is crucial. For this reason, the fitness device 1 of FIG. 1A may rest via at least two different components at a floor. A first component is provided by bottom part 100 of the frame structure 10. The bottom part 100 allows the frame structure 10 to rest on the floor and thus provides a support directly below the display 11. In addition, a stand 2 is provided which allows the frame structure 10 to be positioned inclined to the vertical at the floor and to be nevertheless held in a stable position. The stand 2 extends at an angle to the back side 1B of the frame structure 10 so that a base portion of the stand 2 (formed by a base member 21) is configured to rest on the floor being the bottom part 100 in a specified distance to the bottom part 100.

As further illustrated by FIG. 1B, the stand 2 of this embodiment is designed as a rectangular frame including a base member 21, a crossbar 22 and two parallel lateral bars 20.1, 20.2. The lateral bars 20.1, 20.2 are connected to each other at a first (upper) end via the crossbar 22 and at a second (lower) end via the base member 21. At the crossbar 22 the stand 2 is fixed to a back plate 13 the fitness device 1. The back plate 13 is attached to the frame structure 10 at the back side 1B.

In a display area of the frame structure 10 at which at the display 11 is held, the mounting plate 13 defines a mounting region. At this mounting region the crossbar 22 is attached to the back plate 13 via an attachment portion of the stand 2. The stand 2 is fixed to the back plate 13 (and for example a component of the frame structure 10 covered by the back plate 13) via several fastening elements, for example in the form of screws, bolts or rivets which at least partially extend through the attachment portion at the crossbar 22.

The attachment portion of the crossbar 22 is fixed to the back plate 13 at a location above a recess portion or recess 130 of the back plate 13 and below two grille portions 132.1 and 132.2 for loudspeakers of the fitness device 1. The recess 130 includes an interface region 131. At an edge section of the recess 130 the interface region may accessible for the user and may comprise at least one of a connector for a power supply, a connector for a wired network connection, one or more connectors for a video or sound interface and a main power switch.

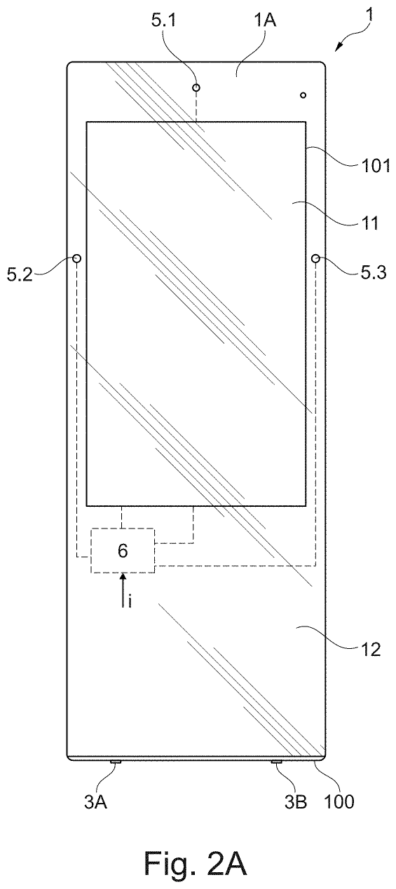

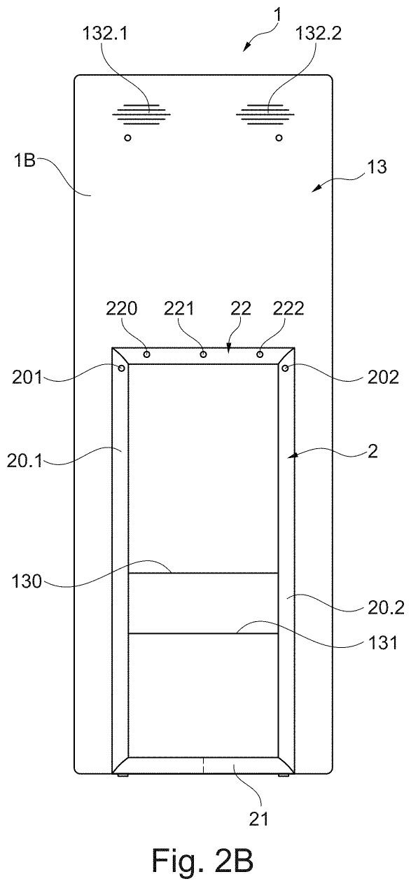

As can be seen from the front view of FIG. 2A, the fitness device 1 may for example in particular include one or more cameras 5.1, 5.2, 5.3 for capturing images of the user facing the front side 1A. For example, a first camera 5.1 may be positioned at the front side 1A in the middle of a portion above the display 11. Additional second and third cameras 5.2 and 5.3 are positioned on lateral sides so that a display recess 101 for a screen of the display 11 is arranged between optics of the second and third cameras 5.2 and 5.3.

The first, second and third cameras 5.1, 5.2 and 5.3 may be part of a camera system of the fitness device 1 which are controlled by a software component running on at least one computing device 6 of the fitness device 1. The first camera 5.1 may be provided for communication, entertainment and/or displaying visual elements, in particular interface elements, depending on a predetermined position of the user. The second and third cameras 5.2 and 5.3 may provide image data for a motion analysis module of the fitness device (implemented by the computing device 6). The motion analysis module may be provided for analyzing position and posture of a user when performing a fitness exercise in front of the fitness device 1. Accordingly, the different cameras 5.1, 5.2 and 5.3 as well as the display 11 are coupled to the computing device 6, and in particular its motion analysis module.

In addition, the computing device 6 may receive input(s) i including a video or stream to be presented on the display 11. The input i may be already pre-processed for displaying the respective content with a holographic effect (explained below in greater detail). Alternatively, the content received as input i may be pre-processed by the computing device 6, for example using artificial intelligence.

In addition, the computing device 6 may include a calibration module recognizing proportion differences between images captured by the second and third cameras 5. and 5.3. Thereby, the calibration module may also be configured to detect at which distance and thus position from the fitness device 1 a user is located. This may result in calibrating the motion analysis module with x and y coordinates which may then also be used for defining at least one of an appearance, a size and a position of at least one visual element to be displayed on the display 11. Hence, visual elements display may be dynamically adapted based on image data provided by the camera system including the second and third cameras 5.2, 5.3. For example, depending on the distance of a user to the fitness device 1 a displayed visual element may be augmented and/or displayed with an increased brightness.

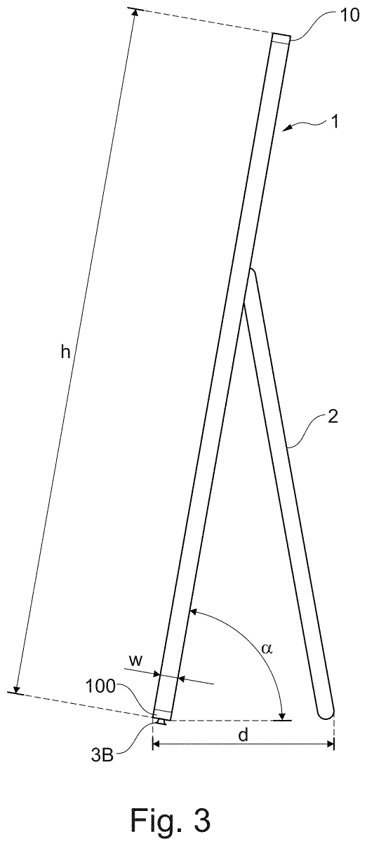

The back view of FIG. 2B further illustrates the position, dimensions and the configuration of the stand 2 with respect to the frame structure 10. Here, a height of the stand 2 is greater than one half of a total height h of the frame structure 10 (cf. also FIG. 3). Thereby, the attachment portion of the crossbar 22 is fastened to the mounting region at the back side 1A at a height (distance to the bottom part 100) which is greater than one half of the total height h of the frame structure 10 but less than two-thirds of the total height.

In the shown example, the crossbar 22 is fixed to the back plate 13 via three fastening elements, for example three screws, at three mounting holes 220, 221, and 222. In addition, the first (upper) end of each lateral bar 20.1, 20.2 is separately fixed to the back plate 13 via mounting holes 201, 202 for providing an additional fixation of the stand 2 and its parts to the back plate 13 and thus to the frame assembly of the fitness device 1.

As can be seen from the side view of FIG. 3, the frame of the frame structure 10 has a total height h which is several times bigger than a width w of the frame. Accordingly, the frame of the frame structure 10 is relatively thin or slim which provides for an elegant outer appearance of the fitness device 1. For example, the width w lies in the range of 2 mm to 5 mm, whereas the total height h is the greater of then 1.5 m, for example lies in the range of 1.5 m to 1.8 m.

The stand 2 extends from its attachment portion at the crossbar 22 to its base member 21 at an angle of about 18° to 25° to the frame structure 10. Thereby, the frame structure 10 (and the display 11) may be held in a stable inclined position. Accordingly, the frame structure 10 does not extend in a completely vertical plane but with a certain inclination to the vertical, namely at an angle α of less than 90° relative to the floor. For example, the angle α lies in the range of 75° to 82°.

The base member 21 of the stand 2 is furthermore resting on the floor at a distance d to the bottom part 100. For example, the distance d lies in the range of 40 cm to 50 cm.

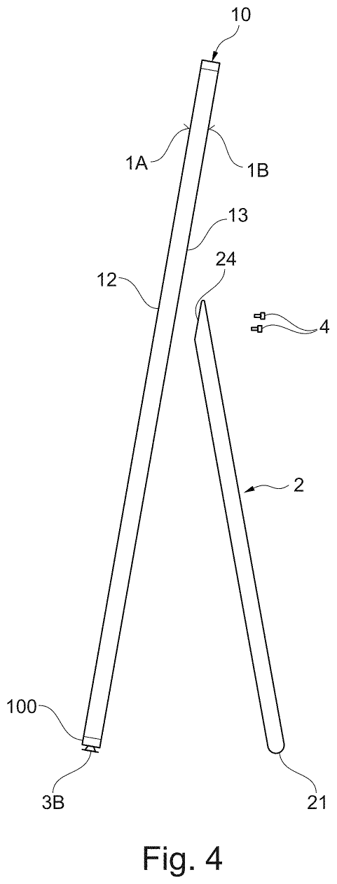

The exploded view of FIG. 4 in particular illustrates an inclined mounting interface of the stand 2 for abutting on the back side 13. Via the inclined mounting interface 22 the stand 2 may abut along a defined plane area against the back plate 13 while the stand 2 being angled away from the back plate 13. FIG. 4 furthermore illustrates screws 4 to be used for fastening the stand 2 via the mounting holes 220, 221, 222 and 201, 202 to the back plate 13 and also to a part of the frame structure 10 covered by the back plate 13.

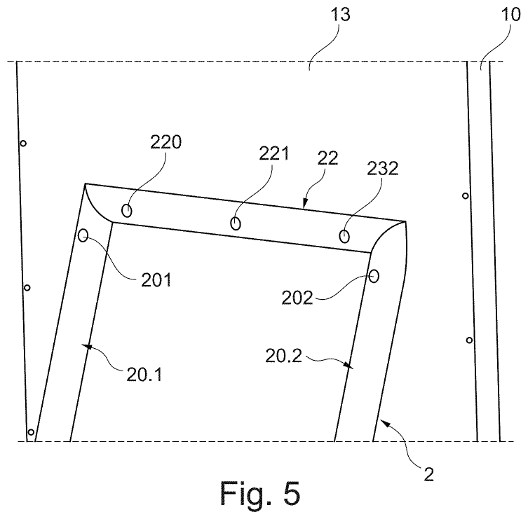

The enlarged view of FIG. 5 shows that the mounting holes 220, 221 and 222 of the crossbar 22 as well as the mounting holes 201 and 202 of the two lateral bars 20.1 and 20.2 in greater detail.

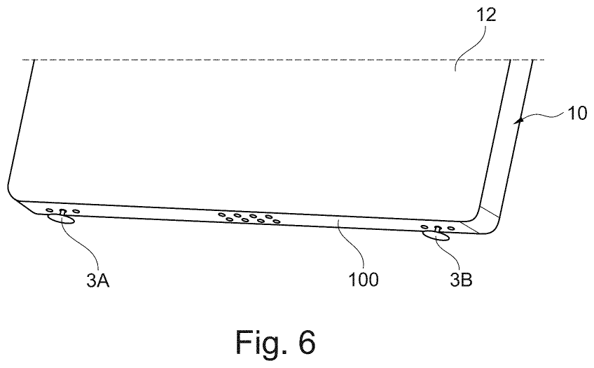

The enlarged view of FIG. 6 furthermore illustrates that the bottom part 100 of the frame structure 10 may include at least two height adjusters 3A, 3B. Via this height adjusters 3A, 3B of the bottom part 100 the frame structure 10 may rest on the floor. By turning the height adjusters 3A, 3B in a threaded hole of the frame structure 10 irregularities of the floor may be compensated for and an inclination of the frame structure may be adjusted.

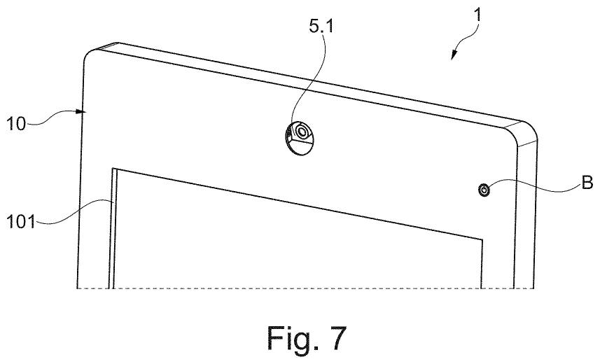

FIG. 7 shows an enlarged view of an upper portion of the fitness device 1. Therefore, FIG. 7 in particular illustrates the arrangement of the first camera 5.1 above the display recess 101. Additionally, FIG. 7 also shows a power switch B on a corner portion at the front side 1A. By pressing the power switch B the fitness device 1 may be switched on or off.

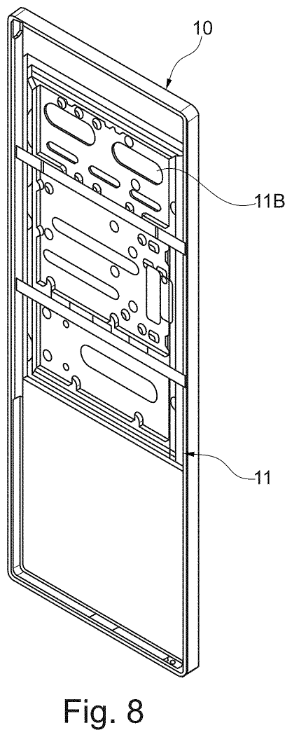

FIG. 8 shows the rectangular frame structure 10 of the fitness device 1 with the display 11 mounted to it. In FIG. 8 a rear surface 11B of the display 11 is visible. The display 11 is fixed within the frame structure 10 at a predefined position and orientation so as to be held in a particular relative position to the mirror.

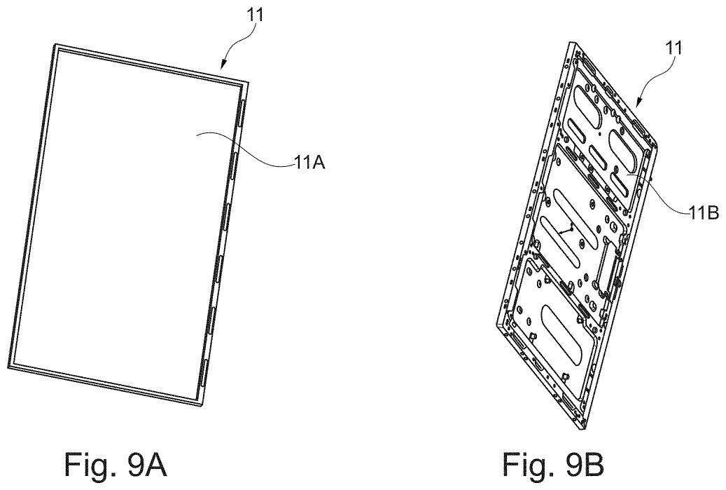

FIGS. 9A and 9B show the display 11 with a front surface 11A and its rear surface 11B, respectively, without the further components of the fitness device 1.

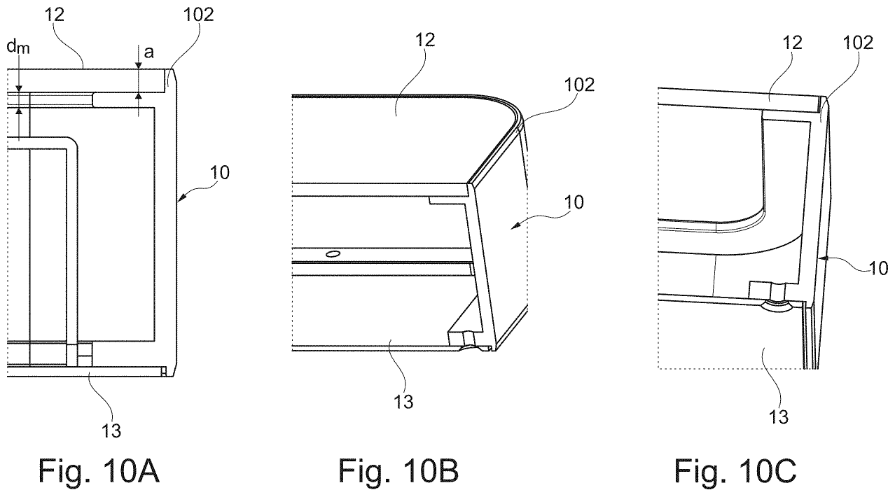

The cross-sectional views of FIGS. 10A, 10B and 10C further illustrate the arrangement of the mirror 12 and the display 11 at the fitness device 1. In the illustrated embodiment, the mirror 12 has a thickness of more than 0.3 millimeters and is provided with a reflective film powder coating for reflecting the incident light from a user. The mirror 12 is held in the frame structure 10 at a mirror enclosure 102 so that the display 11 has it to be fixed at the frame structure 10 behind the mirror 12 at a predefined minimum distance dₘ of at least 1.5 millimetres. In addition, the mirror 12 is provided with a specific transparency-opacity ratio of at least ¼ (up to ¾). Accordingly, the mirror 12 may have a transparency between 20% and 75%. In combination with the display 11 arranged at the predetermined minimum distance dₘ behind the mirror 12 parts of content displayed on the display 11 on a black toned background may be presented with a certain holographic effect. A visual element displayed on correspondingly black tone background having a hue in the range of 0° to 345° in the HSL or HSV color code domain results in an additional projection of the visual element on the rear surface of the mirror 12. Thereby, a duplicate of the visual element is projected and a kind of glow and hollow effect for the displayed visual element is achieved. In this case, by using a black tone background and the reflective surface of the mirror 12 with its predefined transparency-opacity ratio, a light reflection refraction phenomena may be generated so that a user may perceive a displayed (non-black and, compared to the background, brighter) visual element as a kind of floating hologram at the front side 1A of the fitness device 1.

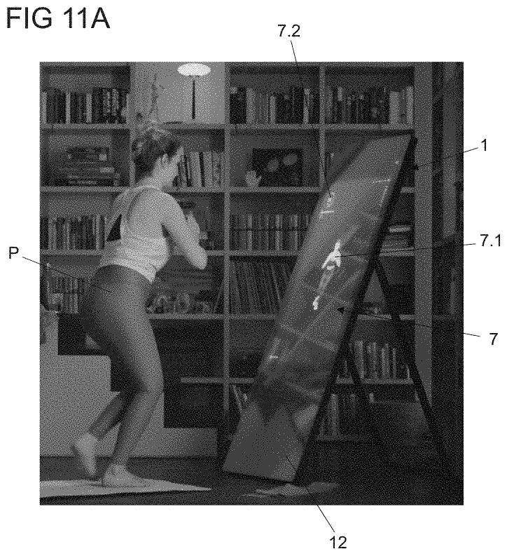

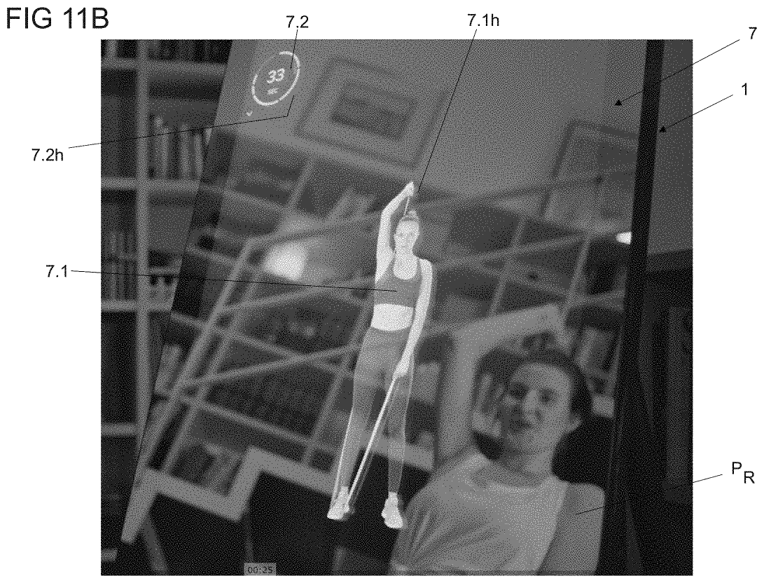

A corresponding holographic effect, which is further illustrated by exemplary embodiments of FIGS. 11A, 11B, 12 and 13, may be altered on the basis of a ratio between the black tone of the background and the transparency of the mirror 12. As a rule of thumb, it may be considered that the lighter the black toned background, the smaller the transparency of the mirror 12 should be in order to provide the user with an agreeable holographic effect. For this reason, at least one processor of the computing device 6, in an exemplary embodiment, may be configured to pre-process content to be displayed, depending on a known/preset transparency-opacity ratio of the mirror 12, in order to black out a background in the content to be displayed and to present at least one visual element on the background (which was identified, by the at least one processor, as a main visual element in the content) so that this visual element appears with a certain hue and brightness on the black tone background.

FIG. 11A shows an exemplary embodiment of the fitness device 1 showing exercise content. The exercise content includes a visual element 7.1 in the form of a visual representation of a trainer showing the exercise to be performed. The visual element 7.1 is displayed as part of a user interface 7 by the display 11. The user interface 7 is displayed on a black toned background, whereas the visual element 7.1—positioned in the center of the display 11—is displayed with lighter and brighter colors having a hue in the range of 0° to 345°. In addition to the virtual trainer of the visual element 7.1 additional visual elements are displayed, like are visual element 7.2 in the left upper corner of the user interface 7 indicating a number of repetitions to be performed. The different visual elements 7.1, 7.2 are displayed such that perception of the visual elements 7.1, 7.2 is facilitated for a user P in front of the fitness device 1.

As can be seen from the enlarged view of the user interface 7 in FIG. 11B, the visual element 7.1 and 7.2 are superimposed on a reflection P_{R} of the user P on the mirror 12. Based on the previously described configuration of the mirror 12 and the display 11 in combination with the black toned background of the user interface 7 and the lighter colors of the displayed visual elements 7.1 and 7.2, a holographic effect is achieved for the displayed visual elements 7.1 and 7.2. In particular, a duplicate image is projected in each case on the rear surface of the mirror 12 so that each visual element 7.1, 7.2 may be perceived by the user P as a floating hologram on the front side 1A. This may include that the visual elements 7.1 and 7.2 are generated with a kind of glow and hollow effect on the black toned background of the user interface 7.

The corresponding holographic effect is used, for example, to augment a virtual or live streamed trainer or avatar as a holographic image while a user P is doing exercises or any other comparable movement for which guidance by the fitness device 1 by a particular visual element might improve the user experience.

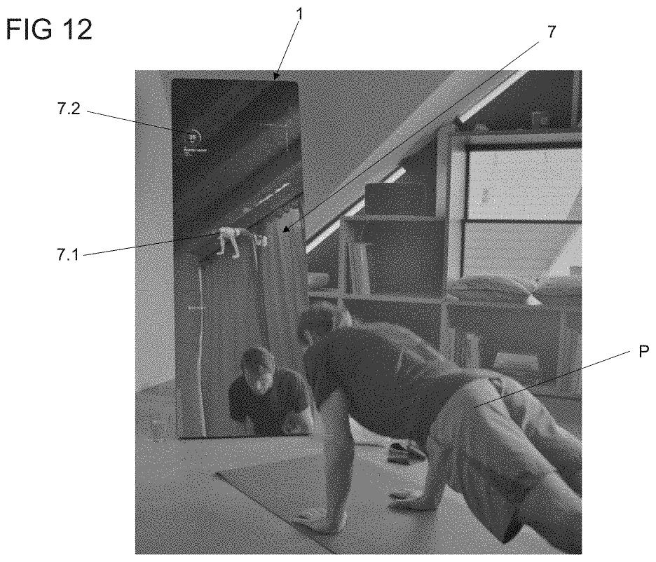

As further illustrated by FIG. 12, the fitness device 1 may dynamically vary at least one of an appearance, a size and a position of at least one of the visual elements 7.1, 0.02 on the user interface 7 depending on the detected decision of the user P in front of the fitness device 1.

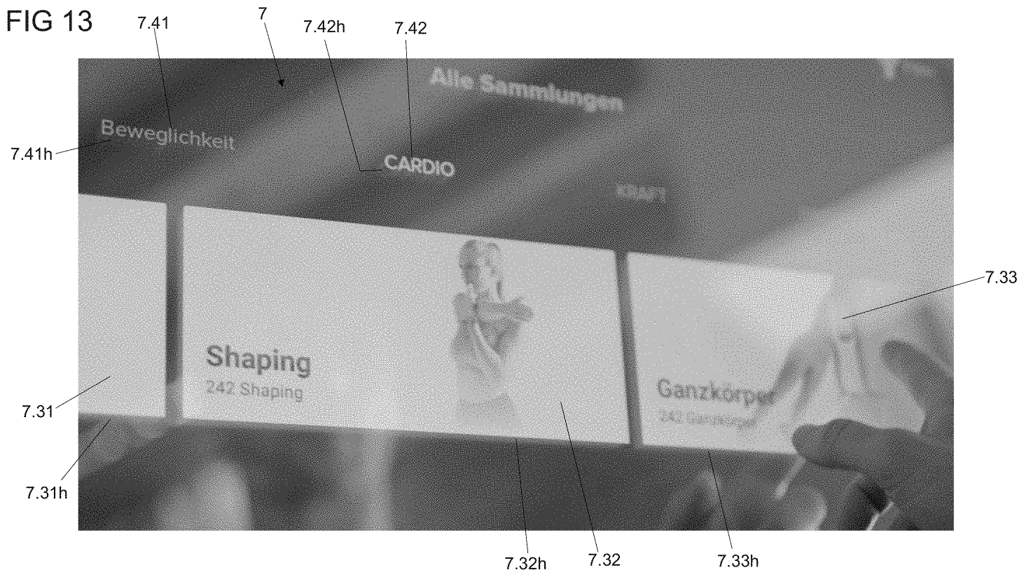

Additionally, visual elements with a holographic effect may be presented on the fitness device 1 as interface elements 7.31, 7.32, 7.33 for providing commands to the computing device 6. For example, different interface elements 7.31, 7.32 and 7.33 may allow a user, by touching a corresponding touch-sensitive portion of the mirror 12 at which the corresponding interface elements 7.31, 7.32 and 7.33 is displayed, to trigger a certain operation event and thus command. Such a command might, for example, cause selecting a certain program or exercise content to be displayed. In the exemplary embodiment of FIG. 13, each of the interface elements 7.31, 7.32 and 7.33 are displayed with the holographic effect to be perceived as a kind of floating hologram on the reflective display surface of the fitness device 1. In addition, visual elements 7.41, 7.41 comprising letters for, for example, describing subprograms, titles or menu items may be presented with duplicate letters on the mirror 12 so as to also achieve a glow and hollow effect and the letters also appearing as a floating hologram.

The visual elements 7.41 and 7.42 and the interface elements 7.31, 7.32 and 7.33 of FIG. 13 are hence presented with a duplicated holographic image 7.41h, 7.42h or 7.31h, 7.32h, 7.33h, respectively. This duplicate is visible for the user P together with the respective visual element displayed on the display 11 behind the mirror 12. A touch input on the displayed interface elements 7.31, 7.32 and 7.33 may for example allow a navigation through different menus. A user P may also be able to navigate across different holographic views. The holographic view may, for example, differ in a ratio between the black tone of the background and a hue of at least a portion of a visual/interface element 7.1, 7.2, 7.31, 7.32, 7.33, 7.41, 7.42.

The computing device 6 of the fitness device 1 may receive information from different network input sources as part of the input i, for example, from at least one other smart device located in a house or gym. Such information may also be used to augment holographic content, in particular, if such information allows for determining a position of the user with respect to the fitness device 1 and/or a potential approach of the user to the fitness device 1.

The fitness device 1 may for example also receive information from a connected car or any comparable vehicle connected to the Internet. The fitness device 1 may also equipped with at least one speaker and/or at least one microphone in order to allow for using the fitness device 1 for videoconferencing. Also, in this context, the described holographic effects for displayed visual elements may be beneficial. For such a functionality it may also be possible to vary at least one of an appearance, a size and a position of at least one visual element depending on a position of the user P in front of the fitness device 1. This may, for example, include augmenting a representation of a remote communication partner in a videoconference depending on the position of the user in front of the fitness device 1.

From the foregoing description it should be clear that the fitness device 1 may in particular include at least one of a microphone for capturing sound inputs, a video camera, a speaker sound system to accompany the visuals presented and a connectivity module, e.g., for a Wi-Fi and/or Bluetooth connection. The connectivity module may, e.g., allow exchange of data and multimedia content via the Internet and/or other local devices. Based on the connectivity module the fitness device 1 may in particular be capable and configured to receive signals from at least one other device, for example from at least one other local or remote fitness device also equipped with a connectivity module.

The foregoing description of the embodiments has been provided for purposes of illustration and description. It is not intended to limit the disclosure. Individual elements or features of a particular embodiment are generally not limited to that particular embodiment, but, where applicable, are interchangeable and can be implemented in another embodiment, even if not specifically shown or described. The same elements may also be varied in one or more ways. Such variations are not to be regarded as a departure from the disclosure, and all such modifications are intended to be included within the scope of the disclosure.

What is claimed is:

1. A device, comprising:
a mirror which reflects incident light from a user facing a front surface of the mirror to present a reflected image of the user and which transmits incident light from a rear surface of the mirror; and
at least one display arranged behind the mirror for displaying content superimposed on at least a portion of the reflected image of the user;
wherein the at least one display is configured to display at least one visual element of the content on a black toned background,
wherein the display and the mirror are configured to create a holographic effect for the at least one visual element by an additional projection of the at least one visual element on the mirror, and
wherein, for generating the additional projection,
a front side of the display at which the content is displayed is arranged at a predetermined minimum distance to the rear surface of the mirror and
the mirror has a predetermined transparency-opacity ratio.

2. The device of claim 1, wherein the at least one display is configured to display at least a portion of the at least one visual element with a hue in the range of 0° to 345° in the HSL or HSV color space on the black toned background.

3. The device of claim 1, wherein the at least one display is configured to display the black toned background with a black or gray color having a color code according to the RGB color code in which r=b=g.

4. The device of claim 4, wherein the at least one display is coupled to a computing device configured to calculate values for r, b and g depending on the transparency-opacity ratio of the mirror.

5. The device of claim 1, wherein the predetermined minimum distance lies in the range of 1.5 mm to 7 mm.

6. The device of claim 1, wherein the transparency-opacity ratio of the mirror is in the range of ¼ to ¾.

7. The device of claim 1, wherein the mirror includes a reflective film powder coating for reflecting the incident light from the user.

8. The device of claim 1, wherein the mirror has a thickness of more than 0.3 mm.

9. The device of claim 1, wherein the at least one visual element defines an interface element for a user interface of the device.

10. The device of claim 1, wherein a capacitive field is applied to at least a portion of the mirror so as to provide the front surface of the mirror with at least one touch-sensitive portion.

11. The device of claim 1, comprising at least one camera for determining a position of the user, wherein the device comprises or is coupled to a computing device which is coupled to the at least one camera and the at least one display and comprises at least one processor and a memory including instructions which, when executed by the at least one processor, cause the at least one processor to
determine, based on image data from the at least one camera, the position of the user in front of the device, and
define at least one of an appearance, a size and a position of the at least one visual element for display at the at least one display depending on the determined position of the user.

12. The device of claim 11, wherein the instructions, when executed by the at least one processor, cause the at least one processor to adjust at least one of the appearance, the size and the position of the visual element based on a change in the determined position of the user.

13. The device of claim 1, wherein the device comprises or is coupled to a computing device comprising at least one processor and a memory including instructions which, when executed by the at least one processor, cause the at least one processor to pre-process content to be displayed by the at least one display of the device, the pre-processing comprising:
identifying at least one main visual element in the content for display, and
blacking out a background of the at least one identified main visual element in the content for display.

14. The device of claim 13, wherein the pre-processing uses artificial intelligence for identifying the at least one main visual element in the content for display and the background to be blackened out.

15. The device of claim 1, comprising a frame structure which defines a continuous frame inside which the mirror is held.

16. The device of claim 1, wherein the mirror has a length of at least 1.0 meter and a width of at least 40 cm.

17. A device, comprising:
a mirror which reflects incident light from a user facing a front surface of the mirror to present a reflected image of the user and which transmits incident light from a rear surface of the mirror; and
at least one display for displaying a user interface superimposed on at least a portion of the reflected image of the user, the display being arranged behind the mirror at a predetermined minimum distance to the rear surface of the mirror;
wherein the at the least one display is configured to display a black toned background and at least one interface element on the black toned background with at least a portion of the interface element having a hue in the range of 0° to 345° in the HSL or HSV color space,
wherein the at least one display and the mirror are configured to create a holographic effect for the at least one interface element by an additional projection of the at least one interface element on the mirror.

18. The device of claim 18, wherein the mirror has a predetermined transparency-opacity ratio.

19. A device, comprising:
a mirror which reflects incident light from a user facing a front surface of the mirror to present a reflected image of the user and which transmits incident light from a rear surface of the mirror; and
at least one display arranged behind the mirror for displaying at least one visual element superimposed on at least a portion of the reflected image of the user;
wherein the at least one display is configured to display the at least one visual element on a black toned background,
wherein the at least one display and the mirror are configured to create a holographic effect for the at least one visual element by an additional projection of the at least one visual element on the mirror, and
wherein, for generating the additional projection,
the mirror has a predetermined transparency-opacity ratio and
the at least one display is configured to display the black toned background with a black or gray color having a color code according to the RGB color code in which r=b=g.

20. The device of claim 19, wherein the at least one display is configured to display the at least one visual element with a hue in the range of 0° to 345° in the HSL or HSV color space on the black toned background.
